(19) United States
(12) Patent Application Publication
(10) Pub. No.: US 2017/0252579 A1
(43) Pub. Date: Sep. 7, 2017

(54) LINEAR ACCELERATOR WITH CERENKOV EMISSION DETECTOR

(71) Applicant: ACCURAY INCORPORATED, Sunnyvale, CA (US)

(72) Inventor: Warren Kilby, Lutterworth (GB)

(21) Appl. No.: 15/057,341

(22) Filed: Mar. 1, 2016

(51) Int. Cl. A61N 5/10; G01T 1/22

(52) U.S. Cl. CPC A61N 5/1071; A61N 5/1081; A61N 5/1083; A61N 5/1075; G01T 1/22

(57) ABSTRACT

A radiation treatment system is described, including a linear accelerator (LINAC), having a housing, to emit a treatment beam to a target location and a Cerenkov emission detector, coupled to the housing of the LINAC, to capture a set of images of optical Cerenkov emission generated at the target location by charged particles of the treatment beam. A method is described including emitting the treatment beam from the LINAC to the target location and capturing, using the Cerenkov emission detector coupled to the LINAC, the set of images of optical Cerenkov emission generated at the target location by the treatment beam.

### TECHNICAL FIELD

Embodiments of the present disclosure relate to a Cerenkov emission detector used in radiation treatment delivery systems and, in particular, to capture a set of images of optical Cerenkov emission generated at a target location by a treatment beam.

### BACKGROUND

A linear accelerator (LINAC) is frequently used in radiation treatment to apply a beam of highly energized particles to a target within a patient. A LINAC may apply one or more treatment beams at one or more angles to a location of the target. Dosimetry is measurement of a dose of radiation received by the target or surrounding parts of the patient. In-vivo dosimetry is the measurement of the dose of radiation delivered to the target or surrounding parts of the patient during treatment. A system of in-vivo dosimetry can be used to determine the amount of radiation delivered by a LINAC to a target or surrounding parts of the patient during treatment.

### BRIEF DESCRIPTION OF THE DRAWINGS

The present disclosure is illustrated by way of example, and not by way of limitation, in the figures of the accompanying drawings.

FIG. 1A illustrates components of a radiation treatment system having a robot based LINAC with a Cerenkov emission detector, in accordance with embodiments of the present disclosure.

FIG. 1B illustrates a radiation delivery system, in accordance with embodiments of the present disclosure.

FIG. 2 illustrates a flow diagram of a method for determining radiation delivered to a target location, in accordance with embodiments of the present disclosure.

FIG. 3 illustrates geometry and vectors of a Cerenkov emission detector and skin surface, in accordance with embodiments of the present disclosure.

FIG. 4 illustrates a flow diagram of a method for increasing the signal-to-noise ratio of optical Cerenkov emission, in accordance with embodiments of the present disclosure.

FIG. 5 illustrates a flow diagram of a method for using multiple Cerenkov emission detectors to determine radiation delivered to a target location, in accordance with embodiments of the present disclosure.

FIG. 6 illustrates systems that may be used in performing radiation treatment, in accordance with embodiments of the present disclosure.

FIG. 7 illustrates an image-guided radiation treatment system, in accordance with embodiments of the present disclosure.

FIG. 8 illustrates an image-guided radiation treatment system, in accordance with embodiments of the present disclosure.

FIG. 9 illustrates a gantry based intensity modulated radiotherapy system, in accordance with embodiments of the present disclosure.

### DETAILED DESCRIPTION

One technique for in-vivo dosimetry is point detector dosimetry at entrance or exit surfaces (e.g., using a diode, a metal-oxide-semiconductor field-effect transistor (MOSFET), a thermosluminescent dosimeter (TLD), film, and so forth), but this technique may not be practical for a treatment using a plurality of treatment beam directions, such as some systems using a LINAC. Another technique for in-vivo dosimetry is transmission dosimetry using an electronic portal imaging device (EPID) or other imaging detector, but this technique may not be practical for a highly non-coplanar treatment workspace. Another technique for in-vivo dosimetry is an implantable dosimeter, which has significant drawbacks and limited clinical utility because of the invasive procedure required.

Described herein are a radiation treatment system and a method for detecting Cerenkov (also known as Cherenkov, Cerenkov, Vavilov, or Wawilow) emission generated at a target or surrounding parts of a patient (hereinafter referred to as a target location) by a treatment beam for in-vivo dosimetry. It should be noted that the detected Cerenkov emission does generally not come from particle interactions within the target itself (which may be deep lying within a patient) but from interactions within superficial tissues near the beam entrance to a patient or beam exit from the patient. Optical Cerenkov emission is generated at the target location when a charged particle of a radiation treatment beam emitted by a LINAC moves in a medium of the target location with a phase speed greater than the speed of light in the medium. A Cerenkov emission detector coupled to the LINAC captures a set of images of the optical Cerenkov emission. A delivered dose of radiation can be determined from the set of images. The delivered dose can be compared to an expected dose of a radiation treatment plan and a difference between the delivered dose and expected dose can be determined or compared to the dose measured for the same patient during different treatment fractions to determine the reproducibility of dose delivery.

The optical Cerenkov emission is relatively weak in comparison to ambient light within the treatment room and radiation noise within the camera, creating a low signal-to-noise ratio. Embodiments of the present disclosure describe methods to increase the signal-to-noise ratio of the optical Cerenkov emission to the ambient light and the radiation noise. In one embodiment, these methods may include decreasing noise of ambient light and radiation noise. In another embodiment, these methods may include improving the signal of optical Cerenkov emission. For example, the Cerenkov emission detector may be mounted at a distal end of the LINAC and may have an incident angle (e.g., angle between skin surface overlying the target location and image axis from lens of the Cerenkov emission detector to the skin surface overlying the target location) that is close to being normal and a minimal distance between the detector and the source of Cerenkov emission.

Embodiments of using a Cerenkov emission detector disposed at a distal end of a LINAC may have advantages such as, for example, removing the risk of detector-LINAC collision, preventing reduction in treatment workspace, and preventing obscuration detector's view of the irradiation beam portal by intervening parts of the patient, LINAC, and/or couch. Other advantages include assisting patient setup by taking images to set table position and rotation and detecting patient motion between images.

In one embodiment described herein, two or more Cerenkov emission detectors coupled to a LINAC may be used. Embodiments of the present disclosure also describe a radiation treatment system and methods to determine a delivered dose of radiation when two or more Cerenkov emission detectors receive images of the optical Cerenkov emission generated at the target location by the treatment beam.

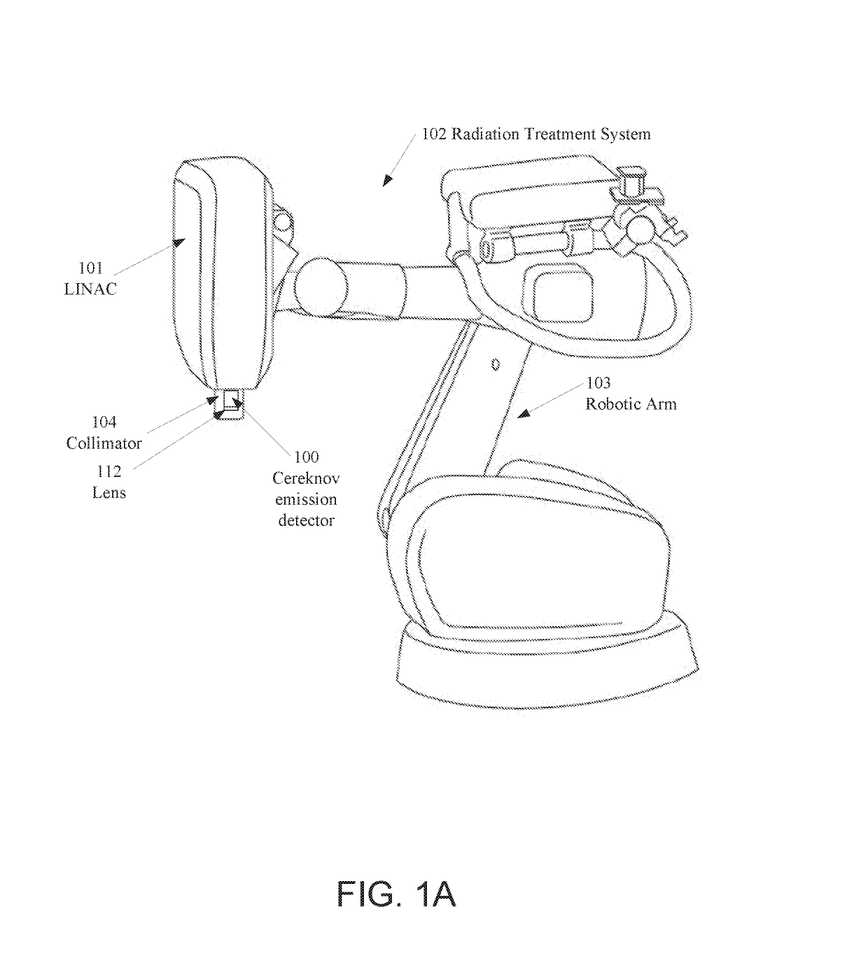

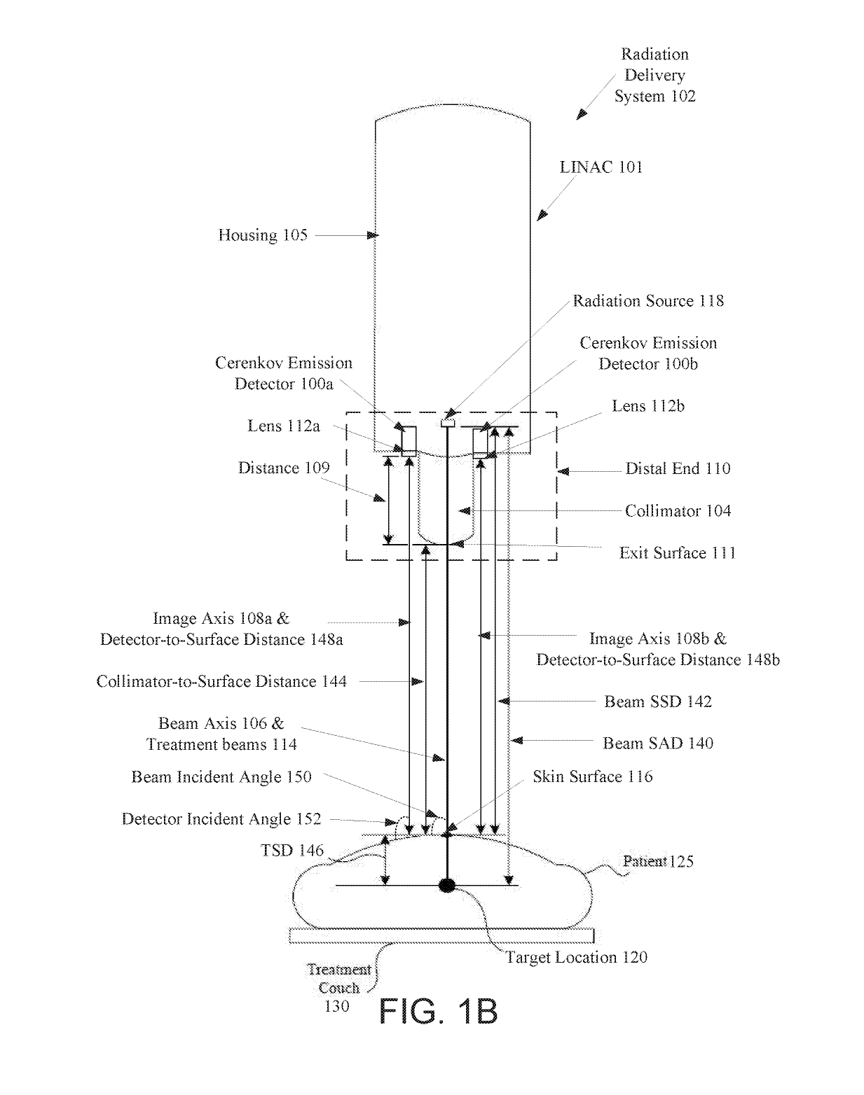

FIG. 1A illustrates components of a radiation treatment system 102 having a robot based linear accelerator (LINAC) 101 with a Cerenkov emission detector 100, in accordance with embodiments of the present disclosure. In one embodiment, the radiation treatment system 102 includes a radiation treatment robot having a LINAC 101 mounted on a robotic arm 103. A Cerenkov emission detector 100 is mounted on a distal end of the LINAC 101 proximate a collimator 104. In one embodiment, the Cerenkov emission detector 100 is a visual light camera. In another embodiment, the Cerenkov emission detector is an infrared camera. In another embodiment, the Cerenkov emission detector is a charge-coupled device (CCD) camera. In another embodiment, the Cerenkov emission detector is an intensified CCD (ICCD) camera. In another embodiment, the Cerenkov emission detector is an electron multiplied ICCD (emICCD) camera (e.g., Princeton Instruments PI-MAX4512 EM). In one embodiment, the Cerenkov emission detector may be operated in a pulsed mode gated by the treatment beam. In another embodiment, two or more Cerenkov emission detectors 100 may be used.

Each Cerenkov emission detector 100 may be designed to be positioned and shielded to maximize the lifetime of each Cerenkov emission detector 100. In one embodiment, each Cerenkov emission detector 100 is positioned at the exit surface of the LINAC 101, to the sides of the treatment beam where each Cerenkov emission detector 100 will be shielded by the collimator 104. In another embodiment, a lens 112 (e.g., Canon EF 135 mm f/2L USM) of each Cerenkov emission detector 100 positioned adjacent to the collimator 104 and is shielded from the treatment beam by the collimator 104. Each lens 112 may be coupled (e.g., using fiber-optics) to remotely positioned Cerenkov emission detector electronics (e.g., optics, an image sensor, an intensifier, and so forth), allowing the Cerenkov emission detector electronics to be positioned at greater distance from the treatment beam. The Cerenkov emission detector electronics may be positioned in a location where space and weight are less restricted to allow greater radiation shielding to be used that at the exit surface of the LINAC 101.

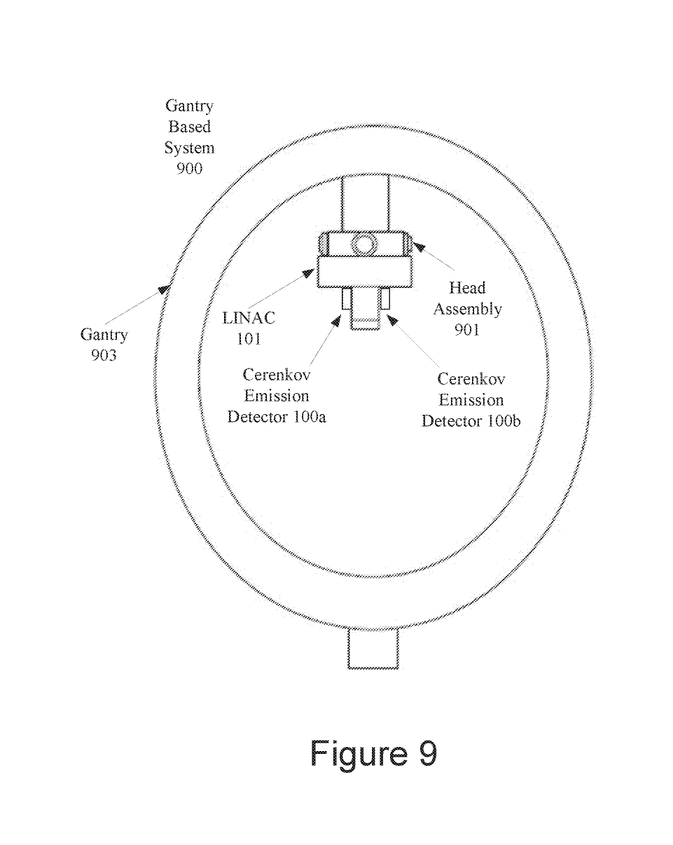

In alternative embodiments, the methods described herein may be used with other types of Cerenkov emission detectors, other types of variable aperture collimators, and other types of radiation treatment systems. In one embodiment, the radiation treatment system 102 is a frameless robotic radiosurgery system (e.g., CyberKnife®). In another embodiment, the radiation treatment system 102 is a gantry-based LINAC treatment system where, for example, LINAC 101 is coupled to a gantry 903 of gantry based system 900 of FIG. 9. Alternatively, other types of radiation treatment systems may be used.

The optical Cerenkov emission at a target location may depend on one or more of skin pigmentation, angle of beam incidence, tissue obliquity, tissue thickness, or other parameters. In one embodiment, the optical Cerenkov emission includes emitted photons that represent energy lost in the first 0-6 mm (e.g., varying with wavelength, varying slightly with skin color, and so forth) of the target location when the treatment beam is emitted to the target location.

FIG. 1B illustrates a radiation delivery system 102, in accordance with embodiments of the present disclosure. The radiation delivery system 102 may include a LINAC 101 that may have a housing 105. The housing 105 of the LINAC 101 may be coupled to a collimator 104. One or more treatment beams 114 may be emitted from a distal end 110 of the LINAC 101 along one or more beam axes 106 to a target location 120. In one embodiment, the target location 120 is located in a patient 125. In another embodiment, the target location 120 is located on a surface of a patient 125. In another embodiment, the target location 120 is located in or on a phantom.

In one embodiment, one or more of the one or more beam axes 106 may be substantially normal to the target location 120 (e.g., perpendicular to the skin surface 116 overlaying the target location, forming a ninety degree beam incident angle 150 with the skin surface 116). The one or more treatment beams 114 may be emitted through an aperture between banks of leaves in the collimator 104.

The collimator 104 may contain any one of various types of collimators (e.g., an iris collimator, a multi-leaf collimator (MLC), and so forth) of different apertures that may be detachably mounted to the LINAC 101. The different collimators may reside in a collimator table, where the radiation treatment robotic may be moved to pick up and drop off collimators based on the collimator type. The particular aperture is matched to the specifics of a radiation treatment plan.

In one embodiment, the distal end 110 of the housing of LINAC 101 may be the radiation source 118. In another embodiment, a distal end 110 of the housing 105 of LINAC 101 may be the area proximate where the housing 105 is coupled to the collimator 104. In another embodiment, a distal end 110 of the housing 105 of LINAC 101 may be the area proximate where the treatment beam 114 is emitted from the housing 105. In another embodiment, the distal end 110 of housing 105 may be where a first Cerenkov emission detector 100a, a second Cerenkov emission detector 100b, and so forth (hereinafter “Cerenkov emission detector 100”) are coupled to the housing 105.

A beam source-to-axis distance (SAD) 140 is measured from the radiation source 118 to the target location 120. A beam source-to-surface (SSD) 142 is measured from the radiation source 118 to the skin surface 116. A collimator-to-surface distance 144 is measured from the exit surface 111 of the collimator 104 to the skin surface 116. A target-to-surface distance (TSD) 146 is measured from the skin surface 116 to the target location 120.

The one or more Cerenkov emission detectors 100 are coupled to the housing 105 of LINAC 101 at locations that do not interfere with the removal and attachment of the collimator 104. In one embodiment, each Cerenkov emission detector 100 may be coupled to the housing 105 at a distal end 110 of the LINAC 101. In another embodiment, each Cerenkov emission detector 100 may include a lens 112 disposed at a distal end 110 of the LINAC 101 proximate exit of the treatment beam from the collimator 104 (e.g., exit surface 111) at a location that does not interfere with removal and attachment of the collimator 104. Each lens 112 may be shielded from the one or more treatment beams 114 by the collimator 104. Each Cerenkov emission detector 100 may capture a set of images (e.g., live images) of optical Cerenkov emission generated at the target location 120 by charged particles of the treatment beam 114 moving in a medium of the target location 120 with a phase speed greater than the speed of light in the medium. The Cerenkov emission detectors 100 may have a first image axis 108a, a second image axis 108b, and so forth (hereinafter “image axis 108”). The first image axis 108a may be from a lens 112a of the first Cerenkov emission detector 100a to the skin surface 116, the second image axis 108b may be from a lens 112b of the second Cerenkov emission detector 100b to the skin surface 116, and so forth. Each image axis 108 may be substantially perpendicular to the skin surface 116 for all, or the majority, of treatment beams included in any treatment plan. The Cerenkov emission detector 100a may have a detector-to-surface distance 148a, the Cerenkov emission detector 100b may have a detector-to-surface distance 148b, and so forth (hereinafter “detector-to-surface distance 148”). The detector-to-surface distance 148 is measured from a lens 112 of the Cerenkov emission detector 100 to the skin surface 116. In one embodiment, each detector-to-surface distance 148 may be substantially equal to the beam SSD 142. In another embodiment, each detector-to-surface distance 148 may be substantially equal to the collimator-to-surface distance 144.

The one or more treatment beams 114 may be emitted from the LINAC 101 from a plurality of treatment beam directions. The detector-to-surface distance 148 may be the same for all of the plurality of treatment beam directions. For example, if a plurality of treatment beams 114 are emitted to a target location 120, and a first subset of the plurality of treatment beams 114 is emitted perpendicular to a horizontal plane (e.g., the treatment couch 130, the floor), a second subset of the plurality of treatment beams 114 is emitted at a 45 degree angle to the horizontal plane, and a third subset of the plurality of treatment beams 114 is emitted parallel to the horizontal plane, the detector-to-surface distance 148 may be the same distance for the first subset, the second subset, and the third subset of treatment beams 114.

In one embodiment, for example, the beam SAD 140 varies between about 80 centimeters (cm) and 100 cm. The exit surface 111 of the collimator 104 may be, for example, 40 cm below the radiation source 118. The TSD 146 (e.g., of a tumor within the patient 125) may be for example, between from 5 cm to ˜30 cm. Accordingly, in one embodiment, the collimator-to-surface distance 144 might vary from 10 cm (limited by collision avoidance between the LINAC 101 and the patient 125) to approximately 55 cm, and the detector-to-surface distance 148 may be approximately the same as the collimator-to-surface distance 144 plus the distance 109 that the Cerenkov emission detector 100 is set back from the exit surface 111 of the collimator 104). In one embodiment, for example, distance 109 may be in a range of 0 cm-30 cm, and therefore the detector-to-surface distance will be less than 85 cm for all beam directions and, in alternative embodiments may be less. When each Cerenkov emission detector 100 is mounted at the distal end 110 of the LINAC 101 (e.g., 0.3-0.5 m typical detector-to-surface distance 148), the signal of the optical Cerenkov emission may be greater (e.g., 29 to 81 times greater) than when the Cerenkov emission detector 100 is mounted at the foot of the treatment couch 130 and above the patient 125 (e.g., 2.7 m from the target location 120).

The first image axis 108a and second image axis 108b may be substantially parallel to each other and substantially normal to (e.g., perpendicular to, a detector incident angle 152 of ninety degrees) the skin surface 116. In one embodiment, a ninety-degree detector incident angle 152 may increase the signal of optical Cerenkov emission by up to 2.5 times compared to a highly oblique detector incident angle 152.

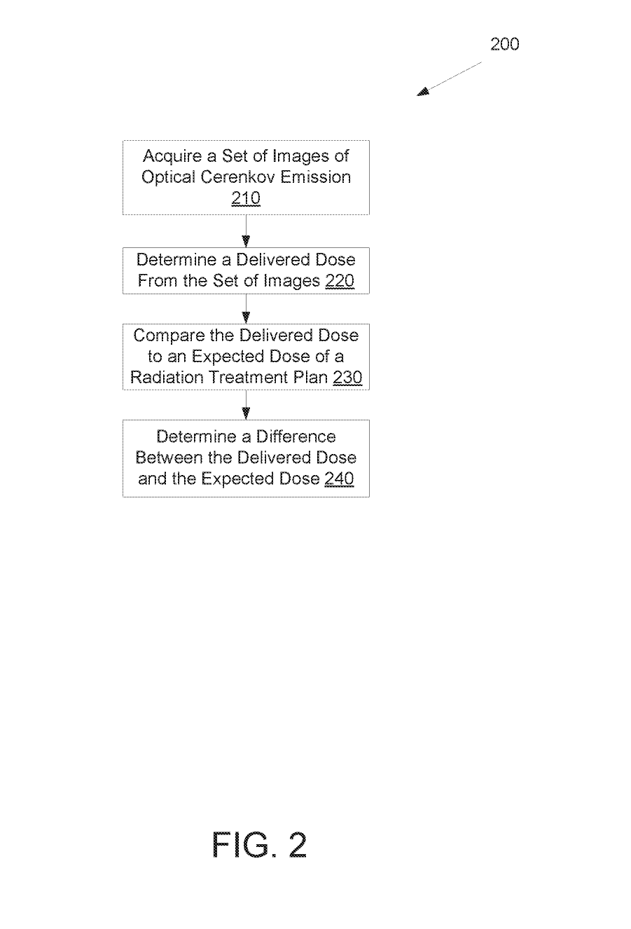

FIG. 2 illustrates a flow diagram of a method 200 for determining radiation delivered to a target location 120, in accordance with embodiments of the present disclosure. Method 200 is described in relation to the determining Cerenkov emission generated at a target location 120 when a LINAC 101 delivers radiation to the target location 120. However, it should be understood that method 200 may also be used to determine radiation delivered to a target location 120 by other systems that emit radiation, in particular, where Cerenkov emission is generated at the target location 120. The method 200 may be performed by processing logic that comprises hardware (e.g., circuitry, dedicated logic, programmable logic, microcode, etc.), software (e.g., instructions run on a processing device to perform hardware simulation), or a combination thereof.

At block 210, processing logic acquires a set of images of optical Cerenkov emission. In one embodiment, the processing logic may capture the set of images using a Cerenkov emission detector 100. The signal-to-noise ratio of the optical Cerenkov emission may be increased by increasing the signal of the optical Cerenkov emission or decreasing the noise (e.g., ambient light, radiation noise, and so forth).

At block 220, processing logic determines a delivered dose from the set of images. The light signal detected in the set of images may be converted to the determined delivered dose. The delivered dose, D, in a column of voxels (e.g., values on a regular grid in three-dimensional space) projected onto each image pixel is related to Cerenkov light intensity emitted from those voxels, I, by some function i.e., the equation below.

D=f(I) (1)

The emitted intensity may not be able to be measured directly. Ignoring any Cerenkov emission detector related measurement perturbations, the emitted light intensity is related to measured light intensity, Im, by the equation below.

I=Im*f1(c)*f2(θ)*f3(d) (2)

The influence parameters at each pixel are skin pigmentation/color, c, angular separation between the viewing angle and the skin surface normal, θ (e.g., incident angle), and distance from the lens of the Cerenkov emission detector to skin surface, d (e.g., detector-to-surface distance 148). In another embodiment, other influence parameters may be used (e.g., tissue obliquity, tissue thickness, and so forth). In turn, these parameters can be used independently in correction functions to remove the effects of superficial tissue optical absorption, f1, optical diffusion within superficial tissue, f2, and divergence of the emitted light in air, f3.

One practical method to determine f(I) would be to irradiate a tissue equivalent phantom at different dose levels in a standard geometry and fit a function to the (D, Im) data pairs obtained, which is equivalent to defining the function in the equation below.

D=f(I/k) (3)

The value of k calculated by the equation below.

k=f1(ccal)*f2(θcal)*f3(dcal) (4)

Here “cal” refers to the calibration phantom and measurement geometry. In general, a measurement made during patient treatment will not correspond exactly to this calibration condition and so in order to apply the calibration in practice it may be necessary to correct each measurement for the effect of the difference in influence parameters between calibration and measurement. This is achieved by combining equations (2) and (4) to calculate I/k from the patient measurement Im at each pixel, i.e., the equation below.

I/k=Im*[f1(c) from m to cal]*[f2(θ) from m to cal]*[f3(d) from m to cal] (5)

Skin pigmentation/color is measured using either the Cerenkov emission detector (in non-intensified mode under standard lighting) or by visual assessment against a standard color scale, and the correction f1 could be achieved with a look-up table or equivalent that is derived from measurements with corresponding phantom materials.

The correction function for the optical diffusion follows Lambert's emission law and therefore the equation below.

f2(θ) from m to cal=(cos(θcal))/(cos(θm)) (6)

The correction function for the optical divergence follows the inverse square law as shown in the equation below.

f3(d) from m to cal=((dm)̂2)/((dcal)̂2) (7)

It is possible to define the angle and distance parameters in the calibration geometry by careful set-up of the beam and phantom alignment. The parameters θm and dm may be determined for each image pixel, for each treatment beam, during patient treatment so that the corrections defined in equations (6) and (7), or the more general forms of them described in equation (5) can be applied. Methods for obtaining these parameters are described next.

The parameters θ and d can be obtained from medical imaging and treatment beam data. Although what follows is related to obtaining these influence parameters for measurements made during patient treatment (θm and dm), they can be equally applied to obtain the influence parameters in the calibration geometry if needed (θcal and dcal). Prior to treatment delivery, it is standard practice to acquire 3D medical imaging data of the patient in the treatment position. The 3D medical imaging data is used during the treatment planning process (e.g., creation of a radiation treatment plan) to define target structures and healthy anatomy, to guide the selection of treatment beam trajectories, aperture shapes, and fluence distributions, to provide physical data on tissue composition for use during radiation dose calculation (e.g., an expected dose of the radiation treatment plan), and to generate digitally reconstructed radiographs for treatment alignment and tracking. As a result of this treatment planning process, the position and orientation of each treatment beam relative to a 3D patient model is defined, together with other information.

The 3D medical imaging data can also be used to accurately calculate θm and dm for each pixel in each treatment beam projection onto the patient skin surface, and the area immediately adjacent, where entrance dose will be measured. A representation of the appropriate skin surface regions (which can be obtained from a 3D medical image in which the air:skin boundary is visualized, such as CT or MRI) and the position of the Cerenkov emission detector and orientation relative to the entrance skin region for each treatment beam can be used. The position and orientation of each Cerenkov emission detector relative to the treatment beam source (e.g., LINAC target, target location, and so forth) and beam central axis (e.g., beam axis) is fixed, due to each Cerenkov emission detector being mounted with a fixed position and orientation within the treatment head. Therefore the position and orientation of the Cerenkov emission detector relative to the patient skin surface for each treatment beam is known from the 3D medical imaging this dataset.

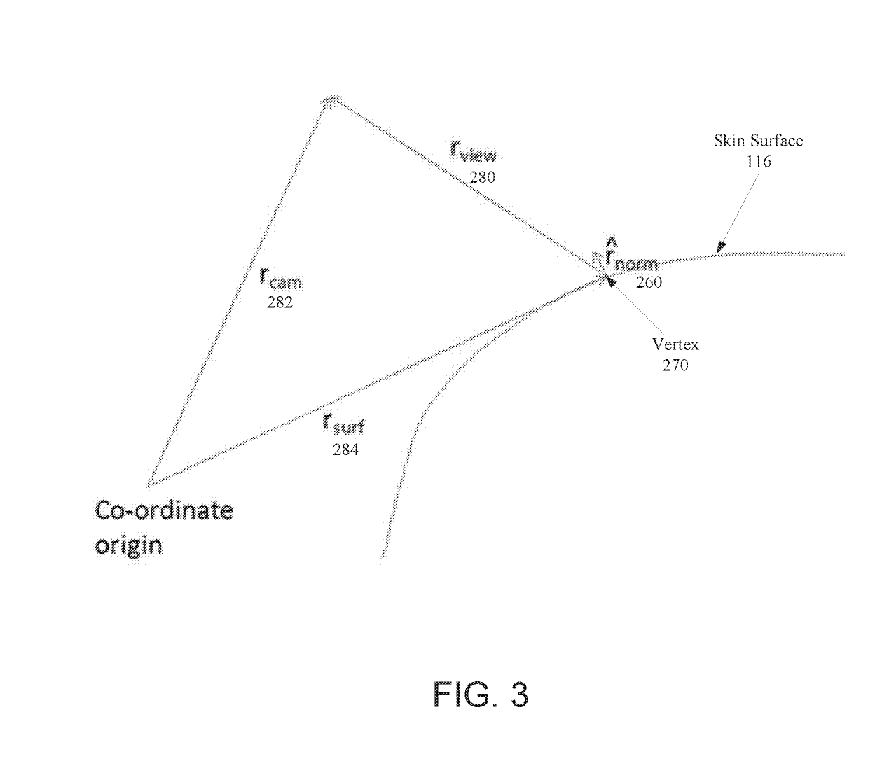

Considering one beam and one point on the skin surface irradiated by that beam, the parameters of interest are the position of the Cerenkov emission detector (or camera focal point) rcam, the position of interest on the skin surface rsurf, the unit skin surface normal vector at that position unit vector rnorm, and the vector linking rsurf to rcam, rview (see FIG. 3).

FIG. 1B illustrates geometry and vectors of a Cerenkov emission detector 100 and skin surface 116, in accordance with embodiments of the present disclosure. FIG. 3 is a simplified 2D representation of the Cerenkov emission detector and skin surface 116 geometry and the relevant vectors that can be used to determine the influence parameters θm and dm. The unit normal vector 260 at each point on the skin surface 116 can be found using multiple methods. For example, if the skin surface 116 is represented as a set of polygons, then the unit normal vector 260 at each vertex 270 is given by the equation below.

unit vector rnorm=(a×b)/∥a×b∥ (8)

In equation (8), a and b are two sides of a polygon originating at the vertex 270. The vector rview 280 and the corresponding unit vector are given simply by combining the known vectors rcam 282 and rsurf 284. The two influence parameters are calculated using the equations below.

θm=arccos(dot product of (unit vector rview) and (unit vector rnorm)) (9)

dm=∥rview∥ (10)

The preceding discussion applies to pre-treatment imaging and generates the influence parameters at the time of treatment planning. However, the same methods could be used to check, and if necessary update the correction terms at the time of treatment delivery if a suitable 3D medical image is acquired immediately before treatment (e.g., cone-beam CT, in-room helical CT, or in-room magnetic resonance (MR)) to assist with patient set-up. The use of this data for treatment alignment requires that the in-room image is registered to the pre-treatment image used for treatment planning, and therefore the complete set of {rcam} from the treatment plan and {rsurf) from the in-room image are available in a consistent co-ordinate system, allowing equations (8)-(10) to be applied at time of treatment and therefore the light signal to be modified according to equation (5) based on the patient imaging at time of treatment. This may be beneficial, as it would be sensitive to changes in patient external shape or position during the time interval between the pre-treatment imaging and treatment delivery. A further extension of this would be to use real-time imaging data acquired during treatment delivery (e.g., from an MR-LINAC system) to correct the light measurement based on the patient-beam geometry at the instant of beam-on.

Returning to FIG. 2, at block 230, processing logic compares the delivered dose to an expected dose of a radiation treatment plan. In one embodiment, the expected dose may be a measured optical Cerenkov emission when a treatment beam is emitted to a phantom (e.g., a water phantom, a plastic phantom, a phantom with fluorescent enhancement, and so forth). In another embodiment, the expected dose may be calculated or looked up in a database. Alternatively the expected dose might be derived from a Cerenkov emission measurement from an earlier treatment fraction (i.e. in order to measure the reproducibility of treatment delivery).

At block 240, processing logic determines a difference between the delivered dose and the expected dose. In one embodiment, if the difference between the delivered dose and the expected dose is below a threshold, the radiation treatment plan is maintained and treatment continues. In another embodiment, if the difference between the delivered dose and the expected dose is above a threshold, at least one of the delivered dose may be decreased, the radiation treatment plan may be reevaluated, or the processing device may prevent the LINAC from emitting more treatment beams. In another embodiment, if the difference between the expected dose and the delivered dose is above a threshold, at least one of the delivered dose may be increased, the radiation treatment plan may be reevaluated, or the processing device may prevent the LINAC from emitting more treatment beams.

It should be noted that the above described operations are just one method of determining the amount of radiation delivered to a target location during treatment and that, in alternative embodiments, certain ones of the operations of FIG. 2 may be optional or take a simpler form.

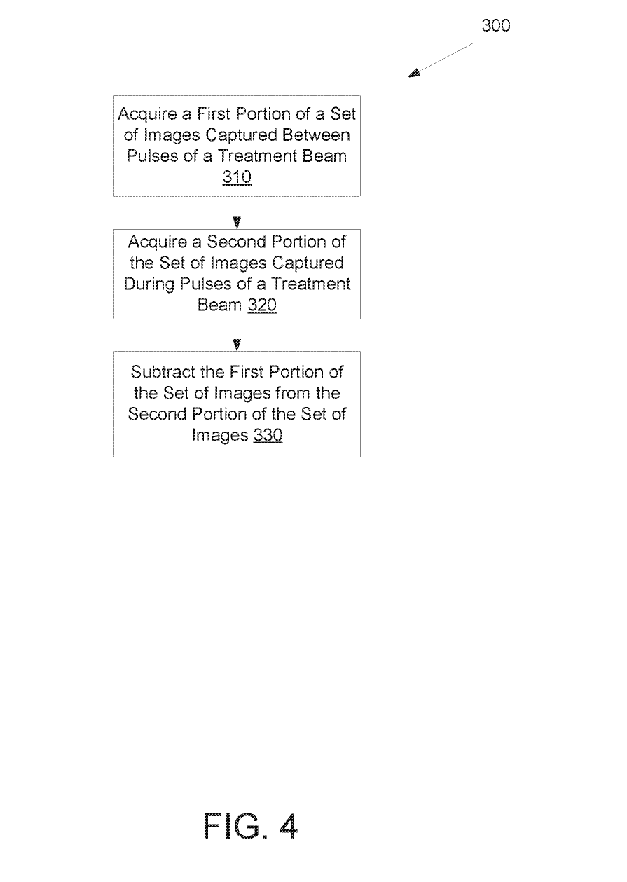

FIG. 4 illustrates a flow diagram of a method 300 for increasing the signal-to-noise ratio of optical Cerenkov emission in which embodiments of the present disclosure may be used. Method 300 is described in relation to removing ambient light from a set of images of optical Cerenkov emission when determining Cerenkov emission generated at a target location when a LINAC 101 delivers radiation to the target location. However, it should be understood that method 300 may also increase the signal-to-noise ratio when radiation is delivered to a target location by other systems that emit radiation, in particular, where Cerenkov emission is generated at the target location. The method 300 may be performed by processing logic that comprises hardware (e.g., circuitry, dedicated logic, programmable logic, microcode, etc.), software (e.g., instructions run on a processing device to perform hardware simulation), or a combination thereof.

At block 310, processing logic acquires a first portion of a set of images captured between pulses of a treatment beam. The first portion of the set of images may provide a measurement of ambient light. In one embodiment, the Cerenkov emission detector 100 may be synchronized with pulses of the treatment beam of the LINAC 101 to capture images between pulses of the treatment beam. In another embodiment, the Cerenkov emission detector 100 may be capable of gated acquisition that is synchronized with gated pulses of the LINAC 101.

At block 320, processing logic acquires a second portion of the set of images captured during pulses of the treatment beam. The second portion of the set of images may provide a measurement of a combination of the ambient light and the optical Cerenkov emission. In one embodiment, the Cerenkov emission detector 100 may be synchronized with pulses of the treatment beam of the LINAC 101 to capture images during pulses of the treatment beam. The ambient light may be maintained constant during pulses and between pulses of the treatment beam of the LINAC 101.

At block 330, processing logic subtracts the first portion of the set of images from the second portion of the set of images. The result of the subtraction may be the optical Cerenkov emission without the ambient light background. In another embodiment, the processing logic calculates an average of first portion of the set of images to determine an average ambient light. The processing logic may subtract the average ambient light from each of the second portion of the set of images to determine optical Cerenkov emission without ambient light background.

It should be noted that the above described operations are just one method of increasing the signal-to-noise ratio of the optical Cerenkov emission in which embodiments of the present disclosure may be used and that, alternatively, certain ones of the operations of FIG. 4 may be optional or take a simpler form.

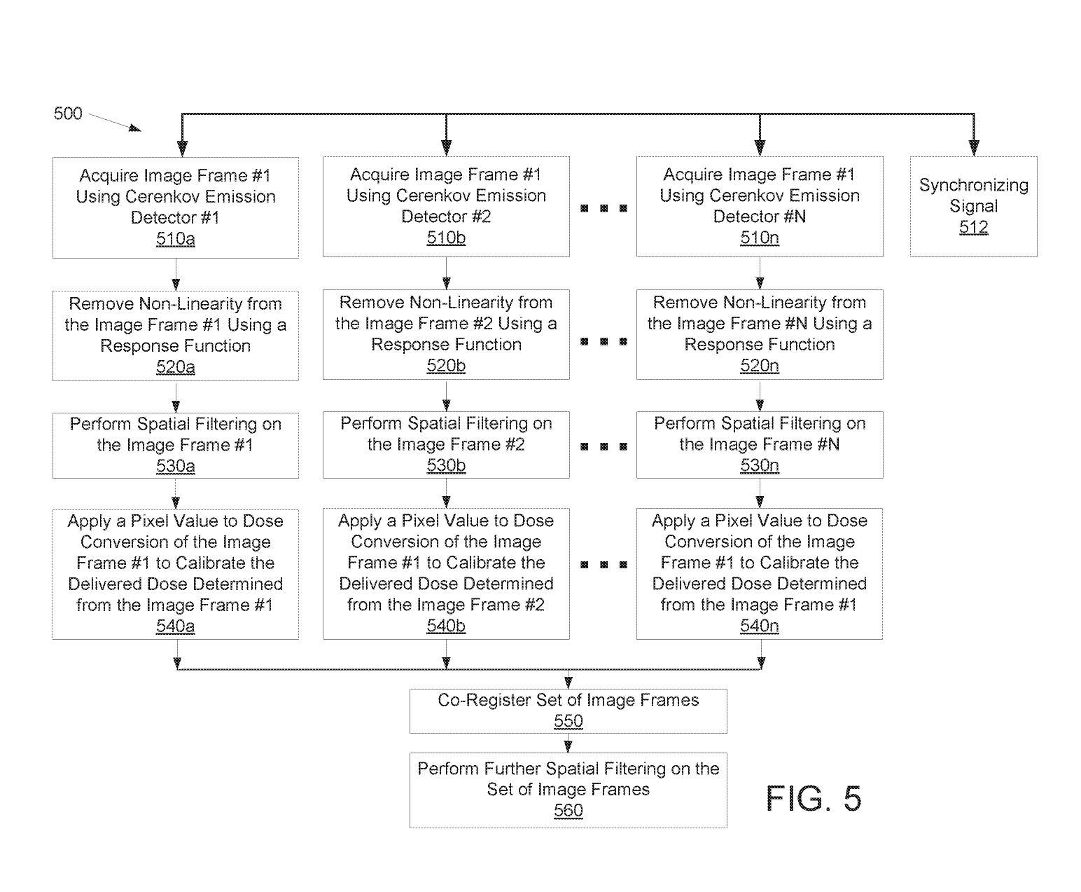

FIG. 5 illustrates a flow diagram of a method 500 for using multiple Cerenkov emission detectors 100 to determine radiation delivered to a target location 120, in accordance with embodiments of the present disclosure. Method 500 is described in relation to the determining Cerenkov emission generated at a target location 120 when a LINAC 101 delivers radiation to the target location 120. However, it should be understood that method 500 may also be used to determine radiation delivered to a target location 120 by other systems that emit radiation, in particular, where Cerenkov emission is generated at the target location 120. The method 500 may be performed by processing logic that comprises hardware (e.g., circuitry, dedicated logic, programmable logic, microcode, etc.), software (e.g., instructions run on a processing device to perform hardware simulation), or a combination thereof.

At block 510, processing logic acquires an image frame using a Cerenkov emission detector 100. In one embodiment, at block 510a processing logic acquires a first image frame using a first Cerenkov emission detector, at block 510b processing logic acquires an image frame at the same point in time using a second Cerenkov emission detector, at block 510n processing logic acquires a corresponding image frame using Cerenkov emission detector #N, and so forth. In another embodiment, the image frame is a single Cerenkov emission detector frame. In another embodiment, the image frame is a summed image generated from multiple frames. In another embodiment, the image frame is a summed image generated from all frames acquired during a single LINAC pulse. In another embodiment, multiple Cerenkov emission detectors (e.g., two or more of the first Cerenkov emission detector, the second Cerenkov emission detector, the Nth Cerenkov emission detector, and so forth) acquire image frames at the same time.

At block 520, the processing logic removes non-linearity (e.g., non-linearity of response of the Cerenkov emission detector) from the image frame using a response function. In one embodiment, a sensor of a Cerenkov emission detector 100 may have a near-linear response to light within its limits, but the linear values may be scaled in the Cerenkov emission detector 100 according to a non-linear function. A response function may be applied to make the values appear linear. In another embodiment, a type of degradation in imaging systems is due to the non-linear response of sensors such as charge-coupled devices (CCD). In one embodiment, if the non-linear response function is known, the inverse of the non-linearity can be applied on each of the pixel values.

At block 530, the processing logic performs a spatial filtering on the image frame. In one embodiment, the spatial filtering removes saturated pixels from the image frame by local median filtering. In another embodiment, the spatial filtering removes dead pixels from the image frame by local median filtering. In another embodiment, the spatial filtering includes linear spatial filtering of local regions within each image. In another embodiment, the spatial filtering includes non-linear spatial filtering (e.g., weighted mean, median filters, and so forth) of local regions within each image. In another embodiment, the spatial filtering includes spatio-temporal filtering (e.g., combining spatial filtering with averaging corresponding pixels in multiple frames) of local regions within each stack of images, assuming multiple frames are acquired at different times. In another embodiment, the spatial filtering includes spectral filtering. In another embodiment, the spatial filtering includes temporal filtering (e.g., averaging corresponding pixels in multiple frames).

At block 540, the processing logic applies a pixel value (e.g., a Cerenkov emission detector pixel value) to dose conversion to the image frame to calibrate the delivered dose determined from the image frame of the Cerenkov emission detector 100. In one embodiment, this conversion is specific to the Cerenkov emission detector 100. In another embodiment, the conversion includes corrections for treatment distance (e.g., image SAD). In another embodiment, the conversion includes corrections for angle of incidence (e.g., angle between image axis and target location). In another embodiment, the conversion includes corrections for skin pigmentation.

At block 550, the processing logic co-registers image frames from a plurality of Cerenkov emission detectors 100. In one embodiment, the processing logic co-registering image frames by transforming the image frames into one coordinate system. In one embodiment, image co-registration is intensity-based. In another embodiment, image co-registration is feature-based. In another embodiment, image co-registration includes affine transformation (e.g., rotation, scaling, translation, and so forth). In another embodiment, image co-registration includes elastic transformations (e.g., locally warping, radial basis functions, and so forth). In another embodiment, image co-registration includes image similarity measures (e.g., cross-correlation, mutual information, sum of squared intensity differences, ratio image uniformity, and so forth).

At block 560, the processing logic performs further spatial and/or spatio-temporal filtering on the set of image frames from the plurality of Cerenkov emission detectors 100. In one embodiment, the further spatial filtering can be performed using corresponding individual pixels or local regions across the set of images.

It should be noted that the above described operations are just one method of determining the amount of radiation delivered to a target location 120 during treatment and that, in alternative embodiments, certain ones of the operations of FIG. 5 may be optional or take a simpler form.

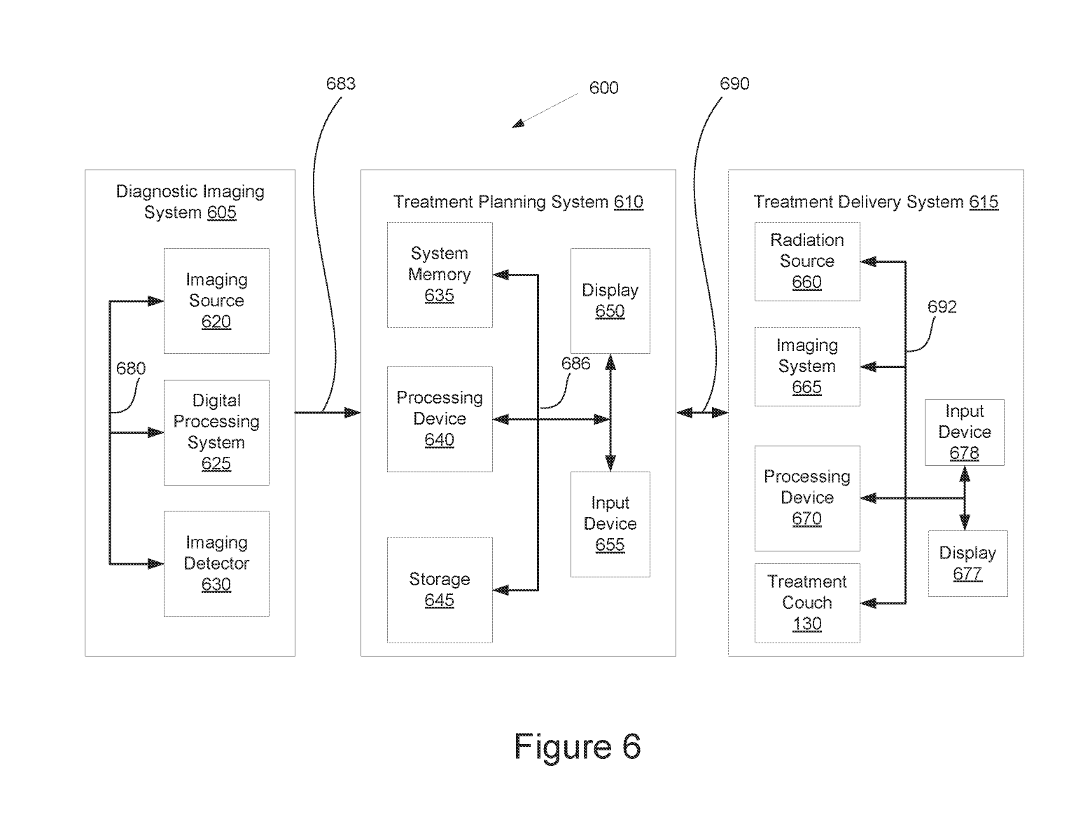

FIG. 6 illustrates systems that may be used in performing radiation treatment, in accordance with embodiments of the present disclosure. These systems may be used to perform, for example, the methods described above. As described below and illustrated in FIG. 6, a system 600 may include a diagnostic imaging system 605, a treatment planning system 610, a treatment delivery system 615 and a motion detecting system (not shown). In one embodiment, the diagnostic imaging system 605 and the motion detecting system are combined into a single unit.

Diagnostic imaging system 605 may be any system capable of producing medical diagnostic images of a patient that may be used for subsequent medical diagnosis, treatment planning, treatment simulation and/or treatment delivery. For example, diagnostic imaging system 605 may be a computed tomography (CT) system, a magnetic resonance imaging (MRI) system, a positron emission tomography (PET) system, or the like. For ease of discussion, diagnostic imaging system 605 may be discussed below at times in relation to an x-ray imaging modality. In other embodiments, other imaging modalities such as those discussed above may also be used.

In one embodiment, diagnostic imaging system 605 includes an imaging source 620 to generate an imaging beam (e.g., x-rays) and an imaging detector 630 (e.g., Cerenkov emission detector) to detect and receive a secondary beam or emission (e.g., Cerenkov emission) stimulated by the beam from the imaging source 620 (e.g., in an MRI or PET scan) or the beam generated by imaging source 620.

In one embodiment, imaging source 620 and imaging detector 630 may be coupled to a digital processing system 625 to control the imaging operation and process image data. In one embodiment, diagnostic imaging system 605 may receive imaging commands from treatment delivery system 615.

Diagnostic imaging system 605 includes a bus or other means 680 for transferring data and commands among digital processing system 625, imaging source 620 and imaging detector 630. Digital processing system 625 may include one or more general-purpose processors (e.g., a microprocessor), special purpose processor such as a digital signal processor (DSP) or other type of device such as a controller or field programmable gate array (FPGA). Digital processing system 625 may also include other components (not shown) such as memory, storage devices, network adapters and the like. Digital processing system 625 may be configured to generate digital diagnostic images in a standard format, such as the Digital Imaging and Communications in Medicine (DICOM) format, for example. In other embodiments, digital processing system 625 may generate other standard or non-standard digital image formats. Digital processing system 625 may transmit diagnostic image files (e.g., the aforementioned DICOM formatted files) to treatment delivery system 615 over a data link 683, which may be, for example, a direct link, a local area network (LAN) link or a wide area network (WAN) link such as the Internet. In addition, the information transferred between systems may either be pulled or pushed across the communication medium connecting the systems, such as in a remote diagnosis or treatment planning configuration. In remote diagnosis or treatment planning, a user may utilize embodiments of the present disclosure to diagnose or treat a patient despite the existence of a physical separation between the system user and the patient.

In one embodiment, treatment delivery system 615 includes a therapeutic and/or surgical radiation source 660 to administer a prescribed radiation dose to a target volume in conformance with a treatment plan. Treatment delivery system 615 may also include imaging system 665 to perform computed tomography (CT) such as cone beam CT, and images generated by imaging system 665 may be two-dimensional (2D) or three-dimensional (3D).

Treatment delivery system 615 may also include a processing device 670 to control radiation source 660, receive and process data from diagnostic imaging system 605 and/or treatment planning system 610, and control a patient support device such as a treatment couch 130. Processing device 670 may be connected to or a part of the camera feedback system described above and operate on the images captured by Cerenkov emission detector 100 of FIG. 1. Processing device 670 may be configured to register 2D radiographic images received from diagnostic imaging system 605, from two or more stereoscopic projections, with digitally reconstructed radiographs (DRRs) generated by digital processing system 625 in diagnostic imaging system 605 and/or DRRs generated by processing device 640 in treatment planning system 610. Processing device 670 may include one or more general-purpose processors (e.g., a microprocessor), a special purpose processor such as a digital signal processor (DSP) or other type of device such as a controller or field programmable gate array (FPGA). The processing device 670 may be configured to execute instructions to perform treatment delivery operations, for example, the method 200 described above in regards to FIG. 2.

In one embodiment, processing device 670 includes system memory that may include a random access memory (RAM), or other dynamic storage devices, coupled to a processing device, for storing information and instructions to be executed by the processing device. The system memory also may be used for storing temporary variables or other intermediate information during execution of instructions by the processing device. The system memory may also include a read only memory (ROM) and/or other static storage device for storing static information and instructions for the processing device.

Processing device 670 may also include a storage device, representing one or more storage devices (e.g., a magnetic disk drive or optical disk drive) for storing information and instructions. The storage device may be used for storing instructions for performing the treatment delivery steps discussed herein. Processing device 670 may be coupled to radiation source 660 and treatment couch 130 by a bus 692 or other type of control and communication interface.

Processing device 670 may implement methods to manage timing of diagnostic x-ray imaging in order to maintain alignment of a target with a radiation treatment beam delivered by the radiation source 660.

In one embodiment, the treatment delivery system 615 includes an input device 678 and a display 677 connected with processing device 670 via bus 692. The display 677 can show trend data that identifies a rate of target movement (e.g., a rate of movement of a target volume that is under treatment). The display 677 can also show a current radiation exposure of a patient and a projected radiation exposure for the patient. The input device 678 can enable a clinician to adjust parameters of a treatment delivery plan during treatment.

Treatment planning system 610 includes a processing device 640 to generate and modify treatment plans and/or simulation plans. Processing device 640 may represent one or more general-purpose processors (e.g., a microprocessor), special purpose processor such as a digital signal processor (DSP) or other type of device such as a controller or field programmable gate array (FPGA). Processing device 640 may be configured to execute instructions for performing simulation generating operations and/or treatment planning operations discussed herein.

Treatment planning system 610 may also include system memory 635 that may include a random access memory (RAM), or other dynamic storage devices, coupled to processing device 640 by bus 686, for storing information and instructions to be executed by processing device 640. System memory 635 also may be used for storing temporary variables or other intermediate information during execution of instructions by processing device 640. System memory 635 may also include a read only memory (ROM) and/or other static storage device coupled to bus 686 for storing static information and instructions for processing device 640.

Treatment planning system 610 may also include storage device 645, representing one or more storage devices (e.g., a magnetic disk drive or optical disk drive) coupled to bus 686 for storing information and instructions. Storage device 645 may be used for storing instructions for performing the treatment planning steps discussed herein.

Processing device 640 may also be coupled to a display device 650, such as a cathode ray tube (CRT) or liquid crystal display (LCD), for displaying information (e.g., a 2D or 3D representation of the VOI) to the user. An input device 655, such as a keyboard, may be coupled to processing device 640 for communicating information and/or command selections to processing device 640. One or more other user input devices (e.g., a mouse, a trackball or cursor direction keys) may also be used to communicate directional information, to select commands for processing device 640 and to control cursor movements on display 650.

Treatment planning system 610 may share its database (e.g., data stored in storage 645) with a treatment delivery system, such as treatment delivery system 615, so that it may not be necessary to export from the treatment planning system prior to treatment delivery. Treatment planning system 610 may be linked to treatment delivery system 615 via a data link 690, which in one embodiment may be a direct link, a LAN link or a WAN link.

It should be noted that when data links 683, 686, and 690 are implemented as LAN or WAN connections, any of diagnostic imaging system 605, treatment planning system 610 and/or treatment delivery system 615 may be in decentralized locations such that the systems may be physically remote from each other. Alternatively, any of diagnostic imaging system 605, treatment planning system 610, and/or treatment delivery system 615 may be integrated with each other in one or more systems.

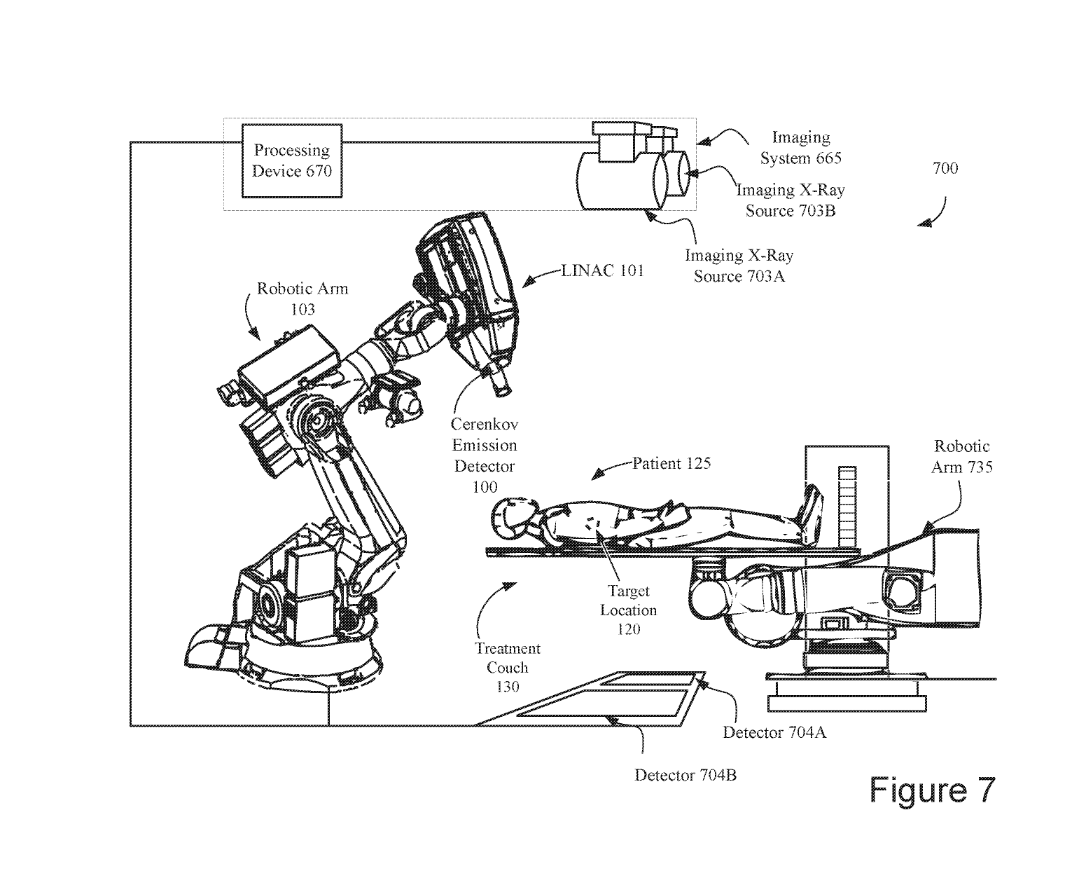

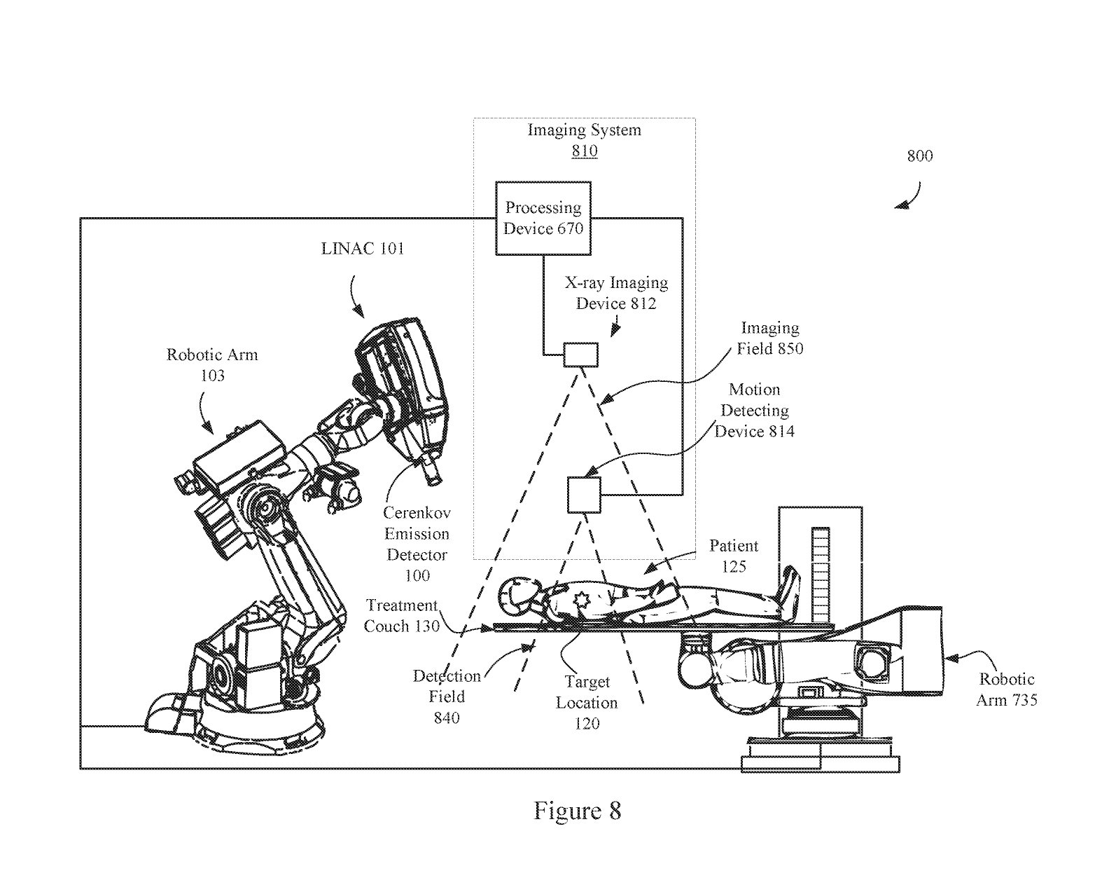

FIGS. 7 and 8 illustrate configurations of image-guided radiation treatment systems 700 and 800, in accordance with embodiments of the present disclosure. In the illustrated embodiments, the radiation treatment systems 700 and 800 include a LINAC 101 that acts as a radiation treatment source, and a Cerenkov emission detector 100. In one embodiment, the LINAC 101 and Cerenkov emission detector 100 are mounted on the end of a robotic arm 103 having multiple (e.g., 5 or more) degrees of freedom in order to position the LINAC 101 to irradiate a pathological anatomy (e.g., target location 120) with beams delivered from many angles, in many planes, in an operating volume around a patient, and to capture images by the Cerenkov emission detector 100 of Cerenkov emission generated in the target location 120 by the beams. Treatment may involve beam paths with a single isocenter, multiple isocenters, or with a non-isocentric approach. Alternatively, other types of image guided radiation treatment (IGRT) systems may be used. In one alternative embodiment, the LINAC 101 and Cerenkov emission detector 100 may be mounted on a gantry based system (e.g., robotic gantry) to provide isocentric beam paths. In one particular embodiment, the IGRT system is the Vero SBRT System (referred to as TM200 in Japan), a joint product of Mitsubishi Heavy Industries Ltd., of Tokyo Japan and BrainLAB AG of Germany, that utilizes a rigid O-ring based gantry.

In one embodiment, the LINAC 101 and Cerenkov emission detector 100 may be positioned at multiple different nodes (predefined positions at which the robot stops and radiation may be delivered) during treatment by moving the robotic arm 103. At the nodes, the LINAC 101 can deliver one or more radiation treatment beams 114 to a target location 120. The nodes may be arranged in an approximately spherical distribution about a patient. The particular number of nodes and the number of treatment beams 114 applied at each node may vary as a function of the location and type of pathological anatomy to be treated. For example, the number of nodes may vary from 50 to 300, or more preferably 15 to 100 nodes and the number of treatment beams 114 may vary from 700 to 3200, or more preferably 50 to 300.

Referring to FIG. 7, radiation treatment system 700, in accordance with one embodiment of the present disclosure, includes an imaging system 665 having a processing device 670 connected with x-ray sources 703A and 703B and fixed x-ray detectors 704A and 704B. Alternatively, the x-ray sources 703A, 703B and/or x-ray detectors 704A, 704B may be mobile, in which case they may be repositioned to maintain alignment with the target location 120, or alternatively to image the target location 120 from different orientations or to acquire many x-ray images and reconstruct a three-dimensional (3D) cone-beam CT. In one embodiment the x-ray sources are not point sources, but rather x-ray source arrays, as would be appreciated by the skilled artisan. In one embodiment, LINAC 101 serves as an imaging source (whether gantry or robot mounted), where the LINAC power level is reduced to acceptable levels for imaging.

Imaging system 665 may perform computed tomography (CT) such as cone beam CT, and images generated by imaging system 665 may be two-dimensional (2D) or three-dimensional (3D). The two x-ray sources 703A and 703B may be mounted in fixed positions on the ceiling of an operating room and may be aligned to project x-ray imaging beams from two different angular positions (e.g., separated by 90 degrees) to intersect at a machine isocenter (referred to herein as a treatment center, which provides a reference point for positioning the patient 125 on a treatment couch 130 during treatment) and to illuminate imaging planes of respective detectors 704A and 704B after passing through the patient 125. In one embodiment, imaging system 665 provides stereoscopic imaging of the target location 120 and the surrounding volume of interest (VOI). In other embodiments, imaging system 665 may include more or less than two x-ray sources and more or less than two detectors, and any of the detectors may be movable rather than fixed. In yet other embodiments, the positions of the x-ray sources and the detectors may be interchanged. Detectors 704A and 704B may be fabricated from a scintillating material that converts the x-rays to visible light (e.g., amorphous silicon), and an array of CMOS (complementary metal oxide silicon) or CCD (charge-coupled device) imaging cells that convert the light to a digital image that can be compared with a reference image during an image registration process that transforms a coordinate system of the digital image to a coordinate system of the reference image, as is well known to the skilled artisan. The reference image may be, for example, a digitally reconstructed radiograph (DRR), which is a virtual x-ray image that is generated from a 3D CT image based on simulating the x-ray image formation process by casting rays through the CT image.

Referring to FIG. 8, in alternative embodiments an imaging system 810 includes a motion detection device 814 to determine target motion, the motion detecting device 814 having a detection field 840. The motion detecting device 814 may detect external patient motion (such as chest movement during respiration) that occurs within an imaging field 850. The motion detecting device 814 can be any sensor or other device capable of identifying target movement. The motion detecting device 814 may be, for example, an optical sensor such as a camera, a pressure sensor, an electromagnetic sensor, or some other sensor that can provide motion detection without delivering ionizing radiation to a patient 125 (e.g., a sensor other than an x-ray imaging system). In one embodiment, the motion detecting device 814 acquires measurement data indicative of target motion in real-time. Alternatively, the measurement data may be acquired at a frequency that is higher (potentially substantially higher) than can be achieved or than is desirable with x-ray imaging (due to ionizing radiation delivered to the patient 125 with each x-ray image). In one embodiment, the motion detecting device 814 does not provide a high absolute position accuracy. Instead, the motion detecting device 814 may provide sufficient relative position accuracy to detect patient movement and/or target movement.

In one embodiment, the motion detecting device 814 is an optical system, such as a camera. The optical system may track the position of light-emitting diodes (LEDs) situated on patient 125. Alternatively, the optical system may directly track a surface region (e.g., skin surface 116) of patient 125, as distinguished from tracking LEDs on the patient 125. There may be a correlation between movement of the target location 120 and movement of the LEDs and/or surface region of the patient 125. Based on the correlation, when motion of the LEDs and/or surface region is detected, it can be determined that the target location 120 has also moved sufficiently to require another diagnostic x-ray image to precisely determine the location of the target location 120.

In another embodiment, the motion detecting device 814 may be the same as the Cerenkov emission detector 100. The motion detecting device 814 captures images of optical Cerenkov emission generated by the treatment beam 114 emitted by the LINAC 101. The Cerenkov emission detector 100 may acquire measurement data indicative of target motion in real-time. In one embodiment, the processing logic may provide a warning or interlock of external patient motion between images. In another embodiment, at blocked nodes the processing logic may provide an option to not interrupt treatment for imaging at if image age expires during or between treatment beams 114.

FIG. 9 illustrates a gantry based (isocentric) intensity modulated radiotherapy (IMRT) system 900, in accordance with embodiments of the present disclosure. In a gantry based system 900, a radiation source (e.g., a LINAC) 101 having a head assembly 901 and Cerenkov emission detectors 100a and 100b (hereinafter “Cerenkov emission detector 100”) are mounted on a gantry 903 in such a way that they rotate in a plane corresponding to an axial slice of the patient 125. Radiation is then delivered from several positions on the circular plane of rotation. In IMRT, the shape of the treatment beam 114 is defined by a collimator 104 (e.g., multi-leaf collimator (MLC)) that allows portions of the beam to be blocked, so that the remaining beam 114 incident on the patient 125 has a pre-defined shape. The resulting system generates arbitrarily shaped treatment beams 114 that intersect each other at the isocenter to deliver a dose distribution to the target location 120. In one embodiment, the gantry based system 900 may be a c-arm based system.

It will be apparent from the foregoing description that aspects of the present disclosure may be embodied, at least in part, in software. That is, the techniques may be carried out in a computer system or other data processing system in response to a processing device 670, for example, executing sequences of instructions contained in a memory. In various embodiments, hardware circuitry may be used in combination with software instructions to implement the present disclosure. Thus, the techniques are not limited to any specific combination of hardware circuitry and software or to any particular source for the instructions executed by the data processing system. In addition, throughout this description, various functions and operations may be described as being performed by or caused by software code to simplify description. However, those skilled in the art will recognize what is meant by such expressions is that the functions result from execution of the code by processing device 670.

A machine-readable medium can be used to store software and data which when executed by a general purpose or special purpose data processing system causes the system to perform various methods of the present disclosure. This executable software and data may be stored in various places including, for example, system memory and storage or any other device that is capable of storing software programs and/or data. Thus, a machine-readable medium includes any mechanism that provides (i.e., stores) information in a form accessible by a machine (e.g., a computer, network device, personal digital assistant, manufacturing tool, any device with a set of one or more processors, etc.). For example, a machine-readable medium includes recordable/non-recordable media such as read only memory (ROM), random access memory (RAM), magnetic disk storage media, optical storage media, flash memory devices, etc.

Unless stated otherwise as apparent from the foregoing discussion, it will be appreciated that terms such as “receiving,” “performing,” “determining,” “forming,” “comparing,” “using,” “subtracting,” or the like may refer to the actions and processes of a computer system, or similar electronic computing device, that manipulates and transforms data represented as physical (e.g., electronic) quantities within the computer system's registers and memories into other data similarly represented as physical within the computer system memories or registers or other such information storage or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, embodiments of the present disclosure are not described with reference to any particular programming language. It will be appreciated that a variety of programming languages may be used to implement embodiments of the present disclosure.

It should be noted that the methods and apparatus described herein are not limited to use only with medical diagnostic imaging and treatment. In alternative embodiments, the methods and apparatus herein may be used in applications outside of the medical technology field, such as industrial imaging and non-destructive testing of materials. In such applications, for example, “treatment” may refer generally to the effectuation of an operation controlled by the treatment planning system, such as the application of a beam (e.g., radiation, acoustic, etc.) and “target” may refer to a non-anatomical object or area.

In the foregoing specification, the disclosure has been described with reference to specific exemplary embodiments thereof. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense.

What is claimed is:

1. A radiation treatment system comprising:
a linear accelerator (LINAC), having a housing, to emit a treatment beam to a target location; and
a Cerenkov emission detector, coupled to the housing of the LINAC, to capture a set of images of optical Cerenkov emission generated at the target location by charged particles of the treatment beam.

2. The radiation treatment system of claim 1, wherein a lens of the Cerenkov emission detector is disposed at a distal end of the LINAC proximate exit of the treatment beam from a collimator, wherein the lens of the Cerenkov emission detector is shielded from the treatment beam by the collimator.

3. The radiation treatment system of claim 2, wherein
the lens of the Cerenkov emission detector is coupled to remotely positioned Cerenkov emission detector electronics of the Cerenkov emission detector,
the remotely positioned Cerenkov emission detector electronics are located at a greater distance from the treatment beam than the lens, and
the remotely positioned Cerenkov emission detector electronics comprise at least one of optics, an image sensor, or an intensifier.

4. The radiation treatment system of claim 1, wherein the Cerenkov emission detector comprises a visual light camera.

5. The radiation treatment system of claim 1, wherein the Cerenkov emission detector comprises an infrared camera.

6. The radiation treatment system of claim 1, wherein the Cerenkov emission detector comprises a charge-coupled device (CCD) camera.

7. The radiation treatment system of claim 1, wherein the Cerenkov emission detector comprises an intensified CCD (ICCD) camera.

8. The radiation treatment system of claim 1, wherein the Cerenkov emission detector comprises an electron multiplied ICCD (emICCD) camera.

9. The radiation treatment system of claim 1, wherein the LINAC is coupled to a robotic gantry.

10. The radiation treatment system of claim 1 further comprising a second Cerenkov emission detector, coupled to the housing of the LINAC, to capture a second set of images of the optical Cerenkov emission generated at the target location by the charged particles of the treatment beam.

11. The radiation treatment system of claim 1, wherein an image axis is from a lens of the Cerenkov emission detector to the target location, wherein the image axis is substantially perpendicular to a skin surface overlaying the target location.

12. The radiation treatment system of claim 1, wherein
a collimator-to-surface distance is measured from a distal end of the LINAC collimator to a skin surface overlaying the target location,
a detector-to-surface distance is measured from a lens of the Cerenkov emission detector to the skin surface overlaying the target location, and
the beam collimator-to-surface distance is substantially equal to the detector-to-surface distance.

13. The radiation treatment system of claim 1 further comprising:
a memory to store the set of images; and
a processing device, operatively coupled to the memory, the processing device to:
determine a delivered dose from the set of images;
compare the delivered dose to an expected dose of a radiation treatment plan; and
determine a difference between the delivered dose and the expected dose.

14. The radiation treatment system of claim 13, wherein the processing device further to:
subtract a first portion of the set of images from a second portion of the set of images, wherein the Cerenkov emission detector captures the first portion of the set of images between pulses of the treatment beam and the Cerenkov emission detector captures the second portion of the set of images during the pulses of the treatment beam;
remove non-linearity of response of the Cerenkov emission detector from each image of the set of images;
remove at least one of saturated pixels or dead pixels from the set of images by local median filtering; and
apply a Cerenkov emission detector pixel value to dose conversion to each image of the set of images, wherein the conversion includes corrections for at least one of distance from a lens of the Cerenkov emission detector to the target location, angle of incidence between the target location and an image axis from the lens of the Cerenkov emission detector to the target location, or pigmentation of the target location.

15. A method comprising:
emitting a treatment beam from a linear accelerator (LINAC) to a target location; and
capturing at an incident angle, using a Cerenkov emission detector coupled to the LINAC, a set of images of optical Cerenkov emission generated at the target location by the treatment beam, wherein a detector-to-surface distance is measured from a lens of the Cerenkov emission detector to a skin surface overlaying the target location and the detector-to-surface distance is less than 85 centimeters.

16. The method of claim 15, further comprising:
maintaining a beam source-to-surface distance (SSD) substantially equal to the detector-to-surface distance, wherein the beam SSD is measured from a distal end of the LINAC to the skin surface overlaying the target location; and
maintaining the incident angle substantially normal to the skin surface, wherein the incident angle is between the skin surface and an image axis from the lens of the Cerenkov emission detector to the target location.

17. The method of claim 15, further comprising increasing a signal-to-noise ratio of the optical Cerenkov emission to ambient light and radiation noise, wherein increasing the signal-to-noise ratio comprises:
subtracting a first portion of the set of images from a second portion of the set of images, wherein the first portion of the set of images is captured between pulses of the treatment beam and the second portion of the set of images is captured during the pulses of the treatment beam;
removing non-linearity of response of the Cerenkov emission detector from each image of the set of images;
removing at least one of saturated pixels or dead pixels from the set of images by local median filtering; and
applying a Cerenkov emission detector pixel value to each image of the set of images, wherein the Cerenkov emission detector pixel value comprises corrections for at least one of the detector-to-surface distance, the incident angle, or pigmentation of the skin surface.

18. A method comprising:
emitting a treatment beam from a linear accelerator (LINAC) to a target location; and
capturing at an incident angle substantially normal to a skin surface overlaying the target location, using a Cerenkov emission detector coupled to the LINAC, a set of images of optical Cerenkov emission generated at the target location by the treatment beam, wherein the incident angle is between the skin surface and an image axis from a lens of the Cerenkov emission detector to the target location.

19. The method of claim 18, further comprising:
maintaining incidence of the treatment beam on the skin surface within an imaging field of view of the Cerenkov emission detector; and
maintaining a beam source-to-surface distance substantially equal to a detector-to-surface distance, wherein the beam source-to-surface distance is measured from a distal end of the LINAC to the skin surface and the detector-to-surface distance is measured from the lens of the Cerenkov emission detector to the skin surface.
